(19) United States
(12) Patent Application Publication
(10) Pub. No.: US 2017/0151278 A1
(43) Pub. Date: Jun. 1, 2017

(54) THERAPEUTIC GAS RELEASING SYSTEM

(71) Applicants: Lorenz MEINEL, Würzburg (DE); Christoph STEIGER, Würzburg (DE)

(72) Inventors: Lorenz MEINEL, Würzburg (DE); Christoph STEIGER, Würzburg (DE)

(21) Appl. No.: 15/317,844

(22) PCT Filed: Jun. 12, 2015
(86) PCT No.: PCT/EP2015/001187

§ 371 Date: Dec. 9, 2016

(30) Foreign Application Priority Data
Jun. 13, 2014 (DE) 10 2014 008 685.2

(51) Int. Cl. A61K 33/24; A61K 9/28; A61K 33/04

(52) U.S. Cl. CPC A61K 33/24; A61K 33/04; A61K 9/28

(57) ABSTRACT

The present invention relates to therapeutic gas releasing systems. The system comprises compounds A and B, wherein A is a therapeutic gas releasing molecule, A and B are not in contact with each other in the therapeutic system during storage, and B enhances therapeutic gas release from A when the therapeutic system is administered to a patient.

### PRIORITY

This application corresponds to the U.S. national phase of International Application No. PCT/EP2015/001187 filed Jun. 12, 2015, which, in turn, claims priority to German Application No. DE 10 2014 008 685.2 filed Jun. 13, 2014, the contents of which are incorporated by reference herein in their entirety.

### FIELD OF THE INVENTION

The present invention relates to therapeutic gas releasing systems. The system comprises compounds A and B, wherein A is a therapeutic gas releasing compound, A and B are not in contact with each other in the therapeutic system during storage, and B is a compound which enhances therapeutic gas release from A when the therapeutic system is administered to a patient.

### BACKGROUND OF THE INVENTION

The influence of gases such as carbon monoxide (CO), nitric oxide (NO) and hydrogen sulfide (H2S) on the body for therapeutic application is a current field of research. For example, Carbon monoxide (CO) was traditionally perceived as a harmful gas. This perception is shifting towards deploying CO for therapeutic purposes (Gibbons et al., Aliment. Pharmacol. Ther., 38 (2013) 689-702). CO is derived from heme oxigenase (HO) activity, degrading heme to CO, iron and biliverdin (Tenhunen et al., Journal of Biological Chemistry, 244 (1969) 6388-6394). Two HO isoforms have physiological relevance, with HO-1 being rapidly responding to various stimuli, including CO (Lee et al., Biochemical and Biophysical Research Communications, 343 (2006) 965-972), chemical or physical stress (Choi et al, Am J Respir Cell Mol Biol, 15 (1996) 9-19). In contrast, HO-2 is constitutively expressed in various tissues, including neurons, liver, kidney and the vascular endothelium (Maines et al., J Biol Chem, 261 (1986) 411-419). CO has beneficial impact on inflammatory conditions including down-regulation of pro-inflammatory proteins such as interleukin 1β or tumor necrosis factor-α, a process which is regulated by mitogen-activated protein kinase (MKK-3)/p38 through the mitogen-activated protein kinase pathway (Otterbein et al., Nat. Med. (N.Y.), 6 (2000) 422-428). Evidence for a disease modifying impact of CO or repair of injury has been collected e.g. in the gastrointestinal arena including inflammation and for diabetic gastroparesis, post-operative ileus, organ transplantation, inflammatory bowel disease and sepsis (Gibbons et al., Aliment. Pharmacol. Ther., 38 (2013) 689-702; Rochette et al. Pharmacology & Therapeutics, 137 (2013) 133-152; Motterlini et al., Nature Reviews Drug Discovery, 9 (2010) 728-743; Babu et al., British journal of pharmacology, (2014)). Target diseases for treatment with CO also include, severe pulmonary arterial hypertension, idiopathic pulmonary fibrosis, chronic inflammation in patients with COPD and prevention of lung inflammation.

Administration of the gasotransmitter NO which is also produced endogenously, is studied for therapeutic potential in acute chest syndrome in sickle cell disease, ischemic reperfusion injury during extended donor criteria liver transplantation, prevention and treatment of bronchopulmonary dysplasia, pulmonary arterial hypertension, hypoxic respiratory failure, tissue perfusion in sepsis, heart transplant placement, acute chest syndrome, respiratory failure in newborn, diabetic food ulcers, chronic lung disease in premature babies, preterm infants with severe respiratory failure, tinea pedis, cutaneous leishmaniasis, respiratory insufficiency, ischemia-reperfusion injury, neuropathic pain, severe malaria, congenital heart defect, idiopathic pulmonary fibrosis, ARDS, endothelial dysfunction, pulmonary hypertension, cardiac transplant rejection, venous ulcers, cystic fibrosis, asthma, eosinophilic esophagitis, pulmonary embolism, hypoxemia, respiratory acidosis, pure autonomic failure and/or bronchiolitis. Administration of H2S is studied for peripheral arterial disease, chronic kidney disease, acute pancreatitis, ulcerative colitis and colonic disease.

The applicability of gases as medicine, however, is hampered by limitations that relate to their storage, precision of dosage and targeting of specific sites of the body. For CO, most studies thus deliver CO gas through the lungs (Hegazi et al., Journal of Experimental Medicine, 202 (2005) 1703-1713; Takagi et al., Digestive Diseases and Sciences, 55 (2010) 2797-2804; Sheikh et al., The Journal of Immunology, 186 (2011) 5506-5513; Uddin et al., Oxidative medicine and cellular longevity, 2013 (2013) Article ID 210563), a mode of administration challenged by a lack of targeting to the affected tissues other than pulmonary. Intestinal insufflation of CO gas was also explored for treatment of certain diseases. However, it requires special equipment, is uncomfortable to the patient and may also lack targeting of specific sites. Local CO delivery was suggested by taking advantage of the gases strong affinity to transition metals, leading to the development of carbon monoxide releasing molecules (CORM) (Onyiah et al., Gastroenterology, 144 (2013) 789-798; Takagi et al., Digestive Diseases and Sciences, 56 (2011) 1663-1671; Motterlini et al., Circ. Res., 90 (2002) e17-e24)). Some patent applications such as WO 2007/085806 thus suggest pharmaceutical formulations containing CORMs for therapeutic gas delivery. Controlled delivery of CO with such systems is, however, a major issue that has to be solved for successful application of such gas releasing compounds in therapeutic settings.

In this respect, WO 2013/127380 suggests carbon monoxide releasing materials which require light to liberate CO. For CORM-2, spontaneous release once in contact with myoglobin or other heme-dependent proteins that trigger dissociation of CO from the metal is described (Rochette et al., Pharmacology & Therapeutics, 137 (2013) 133-152). Carbon monoxide-releasing micelles for immunotherapy were also suggested and it was speculated that thiol compounds and imidazole might induce CO release in the body and that due to lower cysteine levels found in blood plasma as compared to the endosome/lysosome compartment inside of cells, CO would be primarily released from the micelles inside cells (Hasegawa et al, Journal of the American Chemical Society, 132 (2010) 18273-18280). pH-dependent stability of some CO releasing molecules was also suggested to be exploited for tissue specific release of CO. As e.g. AFL186 was found to release CO faster at a pH of 7.4 than at a pH of 2 it was suggested to use such CORM candidate for preferential CO release in the intestine after passing through the acidic stomach (Romão et al., Chem Soc Rev, 2012, 41, 3571-3583).

Hasegawa et al., Romao et al thus relied on endogenous triggers for therapeutic gas release while WO 2013/127380 suggests light to liberate CO so that both documents describe therapeutic systems which are dependent on stimuli which are not part of the pharmaceutical formulation itself.

It can be summarized that present systems for therapeutic gas delivery are limited by control of therapeutic gas release from the system. The problem to be solved by the present invention was thus to provide an improved therapeutic system.

### SUMMARY OF THE INVENTION

The present inventors have found an advantageous therapeutic system comprising a therapeutic gas releasing compound for therapeutic gas delivery, particularly for the delivery of CO, wherein the therapeutic system itself comprises a stimulus which enhances therapeutic gas release from the therapeutic gas releasing compound when the therapeutic system is administered to the patient. This therapeutic system is advantageous over previously described approaches for therapeutic gas delivery as it e.g. does not rely on external stimuli for therapeutic gas release, such as light, pH or the presence of certain molecules in the tissue or inside of cells of the patient to which the therapeutic system is administered.

As therapeutic gas release should not be triggered during preparation of the therapeutic system or during storage, but only when the therapeutic system is administered to the patient, it was found that the stimulus comprised in the therapeutic system should not be in contact with the therapeutic gas releasing molecule comprised in the therapeutic system during storage, thereby increasing storage stability.

The therapeutic system of the present invention is easy and inexpensive to produce and production can easily be upscaled. It is also flexible in terms of the use of different stimuli which can be contained in the therapeutic system rather than synthesizing different therapeutic gas releasing compounds for different applications.

### BRIEF DESCRIPTION OF THE FIGURES

FIG. 1: CO release [ppm] over time [minutes] from aqueous CORM-2 suspension. Na₂SO₃ solution was added as indicated by the arrowhead (n=3).

FIGS. 2A-2C: (2A) Amperometric detection system: (1) Guide tube with glued in cable, (2) Conically tapered joint, (3) CO detector, (4) Ground glass joint, (5) CO sensor, (6) Erlenmeyer flask. (2B) Calibration of CORM-2 (n=1 for 14 independent measurements), expressed as CORM-2 equivalents [mg] (one CORM-2 equivalent represents the amount of CO [ppm] released per milligram CORM-2 when exposed to 25 mL in a 333 mg/L aqueous Na₂SO₃ solution at room temperature). (2C) Calibration of CORM-A1; n=3; dashed lines indicate the 95% confidence interval.

FIGS. 3A and 3B: (3A) CO loss [ppm] in the amperometric detection system over time [hours] following complete release of CORM-2 in 25 mL Na₂SO₃ solution (n=5). (3B) CO release profile using CORM-2 in 25 mL Na₂SO₃ solution plotted as uncorrected, raw data (circles) and corrected data (squares) over time [minutes]. Corrected data was calculated using the CO loss function as shown in A; (n=3).

FIGS. 4A-4D: SEM images of (4A) coated Na₂SO₃ crystals (4B) uncoated Na₂SO₃ crystals (4C) coated Na₂SO₃ crystals within the bulk of the tablet core (asterisks) and the (4D) cellulose acetate coating of the oral carbon monoxide release system OCORS (asterisks). Inserts are magnifications of (4A) coated crystals and (B) uncoated Na₂SO₃ crystals.

FIGS. 5A and 5B: CO release from the oral carbon monoxide release system OCORS in biorelevant media. Tablets were prepared from blends (5A) without citric acid buffer or (5B) containing buffer. CO release was monitored in water (diamonds), simulated gastric fluid (circles) and simulated intestinal fluid (squares). The data was corrected; (n=3).

FIG. 6: CO release from the oral carbon monoxide release system OCORS as a function of tablet shell porosity and number of coated layers in comparison to CORM-2 suspension (open squares; n=3). The oral system was dip coated once (with tenfold pore former; filled circles), fourfold (filled squares) or eightfold (open circles) and the release was monitored and data was corrected for system related CO loss (n=6).

FIG. 7: CO release from CORM-2 suspension [% of total CORM-2 release] was measured in 25 mL of a 333 mg/L aqueous Na₂SO₃ solution at room temperatures a function of ionic strength. The ionic strength was increased with magnesium chloride (black filled circle; n=3) and sodium chloride (open circle; n=3) in comparison to no addition of any salt (grey filled circle, n=9).

### DETAILED DESCRIPTION OF THE PREFERRED EMBODIMENTS

The present invention is as defined in the claims. It is thus directed to a therapeutic system comprising compounds A and B, wherein A is a therapeutic gas releasing compound, A and B are not in contact with each other in the therapeutic system during storage, and B enhances therapeutic gas release from A when the therapeutic system is administered to a patient.

As used in the specification and claims, the singular forms “a”, “an” and “the” include plural references unless the context clearly dictates otherwise.

Preferably, compound A is a CO releasing molecule (CO-RM) in the present invention. In the present invention, compound A is e.g. a metalcarbonyl compound, an aldehyde, an oxalate, a boronocarboxylate, a metal organic framework (MOF) loaded with a therapeutic gas or a silacarboxylate.

In one embodiment, compound A is a metal carbonyl compound comprising a complex of an element of the group of Rh, Ti, Os, Cr, Mn, Fe, Co, Mo, Ru, W, Re, Ir, B and C. More preferably, compound A is a metal carbonyl compound comprising a complex of an element of the group of Rh, Mo, Mn, Fe, Ru, B and C, even more preferably of the group of Rh, Fe, Mn, B and C. The metal carbonyl compounds may be regarded as complexes, because they comprise CO groups coordinated to a metal centre. However the metal may be bonded to other groups by other than coordination bonds, e.g. by ionic or covalent bonds. Thus groups other than CO which form part of the metal carbonyl compound need not strictly be “ligands” in the sense of being coordinated to a metal centre via a lone electron pair, but are referred to herein as “ligands” for ease of reference.

Thus, the ligands to the metal may all be carbonyl ligands. Alternatively, the carbonyl compound may comprise at least one ligand which is not CO. Ligand which are not CO are typically neutral or anionic ligands, such as halide, or derived from Lewis bases and having N, P, O or S or a conjugated carbon group as the coordinating atom(s). Preferred coordinating atoms are N, O and S. Examples include, but are not limited to, sulfoxides such as dimethylsulfoxide, natural and synthetic amino acids and their salts for example, glycine, cysteine, and proline, amines such as NEt3 and H2NCH2CH2NH2, aromatic bases and their analogues, for example, bi-2,2′-pyridyl, indole, pyrimidine and cytidine, pyrroles such as biliverdin and bilirubin, drug molecules such as YC-1 (2-(5′-hydroxymethyl-2′-furyl)-1-benzylindazole), thiols and thiolates such as EtSH and PhSH, chloride, bromide and iodide, carboxylates such as formate, acetate, and oxalate, ethers such as Et2O and tetrahydrofuran, alcohols such as EtOH, and nitriles such as MeCN. Other possible ligands are conjugated carbon groups, such as dienes, e.g. cyclopentadiene (C5H5) or substituted cyclopentadiene. The substituent group in substituted cyclopentadiene may be for example an alkanol, an ether or an ester, e.g. —(CH2)nOH where n is 1 to 4, particularly —CH2OH, —(CH2)nOR where n is 1 to 4 and R is hydrocarbon preferably alkyl of 1 to 4 carbon atoms and —(CH2)nOOCR where n is 1 to 4 and R is hydrocarbon preferably alkyl of 1 to 4 carbon atoms. The preferred metal in such a cyclopentadiene or substituted cyclopentadiene carbonyl complex is Fe.

Preferably, CORM-1, CORM-2, CORM-3, or CORM-401 is used as compound A in the present invention. In a preferred embodiment, the present invention thus relates to a therapeutic system comprising compounds A and B, wherein A is CORM-2, A and B are not in contact with each other in the therapeutic system during storage, and B enhances therapeutic gas release from A when the therapeutic system is administered to a patient.

In one embodiment, an aldehyde according to formula I

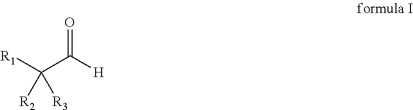

is used as compound A comprised in the therapeutic system of the present invention, wherein R₁, R₂ and R₃ are each independently selected from alkyl, substituted alkyl, cycloalkyl, substituted cycloalkyl, heterocyclyl, substituted heterocyclyl, alkylheterocyclyl, substituted alkylheterocyclyl, alkenyl, substituted alkenyl, aryl, substituted aryl, heteroaryl, substituted heteroaryl, alkylaryl, substituted alkylaryl, wherein the number of C atoms is 1-12 or 1-6 in each case hydroxy, alkoxy, amino, alkylamino, mercapto, alkylmercapto, aryloxy, substituted aryloxy, heteroaryloxy, substituted heteroaryloxy, alkoxycarbonyl, acyl, acyloxy, acylamino, alkylsulfonyl, alkylsulfinyl, F, Cl, Br, NO₂ and cyano; or two or more of R₁, R₂ and R₃ are taken together to form a substituted or unsubstituted carbocyclic or heterocyclic ring structure or an derivative thereof. For any substituent the number of C atoms is 1-12 or 1-6.

A derivative of a compound of formula I being an acetal, hemiacetal, aminocarbinol, aminal, imine, enaminone, imidate, amidine, iminium salt, sodium bissulfite adduct, hemimercaptal, dithioacetal, 1,3-dioxepane, 1,3-dioxane, 1,3-dioxalane, 1,3-dioxetane, α-hydroxy-1,3-dioxepane, α-hydroxy-1,3-dioxane, α-hydroxy-1,3-dioxalane, α-keto-1,3-dioxepane, α-keto-1,3-dioxane, α-keto-1,3-dioxalane, α-keto-1,3-dioxetane, macrocyclic ester/imine, macrocyclic ester/hemiacetal, oxazolidine, tetrahydro-1,3-oxazine, oxazolidinone, tetrahydro-oxazinone, 1,3,4-oxadiazine, thiazolidine, tetrahydro-1,3-thiazine, thiazolidinone, tetrahydro-1,3-thiazinone, imidazolidine, hexahydro-1,3-pyrimidine, imidazolidinone, tetrahydro-1,3-pyrimidinone, oxime, hydrazone, carbazone, thiocarbazone, semicarbazone, semithiocarbazone, acyloxyalkyl ester derivative, O-acyloxyalkyl derivative, N-acyloxyalkyl derivative, N-Mannich base derivative or N-hydroxymethyl derivative can also be used as compound Oxazolidine, thiazolidine, imidazolidinone or oxazolidinone can also be used as compound A in the present invention.

More preferably, as compound A is tri methylacetaldehyde, 2,2-dimethyl-4-pentenal, 4-ethyl-4-formyl-hexanenitrile, 3-hydroxy-2,2-dimethylpropanal, 2-formyl-2-methyl-propylmethanoate, 2-ethyl-2-methyl-propionaldehyde, 2,2-dimethyl-3-(p-methylphenyl)propanal or 2-methyl-2-phenylpropionaldehyde is used herein.

In one embodiment an oxalate, an oxalate ester or amide is used as compound A comprised in the therapeutic system of the present invention.

In one embodiment, carboxyborane, a carboxyborane ester or a carboxyborane amide is used as compound A in the present invention. Preferably,

BHₓ(COQ)_{y}Z_{z}

is used wherein x is 1, 2 or 3, y is 1, 2 or 3, z is 0, 1 or 2, x+y+z=4, each Q is O⁻, representing a carboxylate anionic form, or is OH, OR, NH₂, NHR, NR₂, SR or halogen, where the or each R is alkyl (preferably of 1 to 4 carbon atoms), each Z is halogen, NH₂, NHR′, NR′₂, SR′ or OR′ where the or each R′ is alkyl (preferably of 1 to 4 carbon atoms). More preferably, z is 1 and/or y is 1 and/or x is 3. In one embodiment, at least one Q is O⁻ or OR and the composition includes at least one metal cation, wherein the metal cation is preferably an alkali metal cation or an earth metal cation.

When a boronocarboxylate is used as compound A it is most preferably Na2(H3BCO2) also known as CORM-A1.

In one embodiment of the present invention, a metal organic framework loaded with a therapeutic gas is used as compound A. Metal-organic frameworks (MOFs) are coordination polymers with an inorganic-organic hybrid frame comprising metal ions and organic ligands coordinated with the metal ions. In one embodiment, compound A is a MOF loaded with at least one Lewis base gas chosen from the group comprising of NO, CO and H2S, such as MIL-88B-FE or NH2-MIL-88B-Fe. In another embodiment compound A comprised in the therapeutic system of the present invention is a MOF loaded with at least one Lewis base gas chosen from the group comprising NO, CO and H2S as described in WO2009133278 A1, particularly as described in claims 1 to 13 therein to which it is explicitly referred.

Compound B used in present invention enhances (triggers) gas release from A when the therapeutic system is administered to the patient. Enhanced therapeutic gas release is defined as an increase in the amount of gas released per time by at least 10% as compared to gas release observed without the co-delivery of B and for any time point between the 10% and 90% boundaries of total releasable gas (in theory) from the respective therapeutic system in an aqueous system, such as water or a (simulated) body fluid. Preferably, the enhancement is as such that by co-delivery of compound B in the therapeutic system therapeutic gas release within 300 min is increased by at least 50% as compared to a therapeutic system without co-delivery of compound B in an aqueous medium.

Compound B is preferably a carbonyl substituting ligand, a sulfur-compound, an acid or base, an enzyme, a redox reagent, or a light-emitting compound. The sulfur compound is e.g. an alkali metal or alkaline-earth metal salt, preferably a sodium salt, of sulfite, dithionite, or metabisulfite, or a compound bearing at least one thiol moiety, such as cysteine or glutathione.

For a metal carbonyl compound as compound A and a sulfur compound or other electron withdrawing compound as compound B it is believed that, when this compound B comes in contact with the metal carbonyl compound, a ligand substitution takes place thereby triggering CO release.

In a preferred embodiment of the present invention compound A is a metal carbonyl compound and compound B is a sulfur compound. More preferably, compound A is a metal carbonyl compound wherein at least one ligand is not CO and compound B is a sulfur compound.

A and B are not in contact with each other in the therapeutic system during storage. It has been found that this spatial separation can help to avoid a premature release of the gas from the gas releasing molecule which is a danger even in a solid therapeutic system, such as a tablet, during storage. By storage the time is meant between the finishing of the primary packaging of the therapeutic system and the administration of the therapeutic system to the patient.

When the therapeutic system is administered to the patient a reaction between compounds A and B is no longer hampered by their spatial separation. Compounds A and B preferably come in contact with each other. This means that a reaction between compounds A and B is no longer hampered by their spatial separation. E.g. a substituting ligand for a metal carbonyl compound can then replace an original ligand of the metal carbonyl compound and thereby initiate CO release from the metal carbonyl compound.

In one embodiment the therapeutic system of the present invention is a system wherein A and B are comprised in the therapeutic system as A particles and B particles and contact of A particles with B particles is prevented in the therapeutic system during storage by a coating around the A and/or B particles. A particles do not comprise any compound B, and B particles do not comprise any A, but of course, the particles may comprise other excipients. The coating of the B particles e.g. dissolves by contact with the patient's body fluids when the therapeutic system is administered to the patient thereby granting access of solvent (body fluid) to B and thus “liberating” B and allowing contact with A, which in return induces gas release from A.

Preferably, the therapeutic system of the present invention is for oral administration. Oral administration is a more convenient route of administration than e.g. pulmonary administration. Another advantage of oral administration of therapeutic gases over pulmonary delivery is seen in a lower sensitivity to local high concentrations of therapeutic gases of the gastrointestinal epithelium as compared with the lung epithelium. Oral administration is also favorable in terms of safety aspects over parenteral administration. This is because oral daily administration is well tolerated by patients while daily parenteral administration is not. If daily parenteral administration is circumvented by parenterally administering a controlled release system only once a week which can release a higher amount of therapeutic gas in total, but which should release only a low dose of therapeutic gas every day and for some reasons dose dumping occurs, this poses a safety risk.

The therapeutic system of the present invention is preferably a tablet, a capsule or a granulate. Most preferably it is a tablet or capsule. In one embodiment the therapeutic system of the present invention is a tablet or capsule, and wherein A is a CO releasing molecule and B is a sulfur compound.

The present invention also relates to a tablet or capsule comprising compounds A and B wherein A is a CO releasing molecule and B is a sulfur compound, and wherein A and B are not in contact with each other.

The present invention also relates to a tablet or capsule comprising compounds A and B wherein A is a CO releasing molecule and B is a sulfur compound, and wherein A and B are comprised in the tablet or capsule as A particles and B particles and contact of A with B is prevented in the tablet or capsule by a coating around the A and/or B particles.

Preferably, the therapeutic system of the present invention is coated. This is particularly the case for the therapeutic system being a tablet or capsule.

In one embodiment, the tablet or capsule does not disintegrate completely upon administration to the patient. The tablet or capsule preferably comprises a semipermeable shell. This shell essentially retains the outer dimensions of the tablet or capsule upon transit through the gastrointestinal tract so that the tablet or capsule is excreted in a substantially intact form in the feces after administration to the patient. A micro-environment is created inside the tablet or capsule which favors therapeutic gas release upon administration to the patient.

In one embodiment, the therapeutic system of the present invention is for use in the treatment of an inflammatory disease, preferably an inflammatory gastric or intestinal disease. The therapeutic system of the present invention is thus e.g. for use in the treatment of inflammatory bowel disease, Morbus Crohn, gastritis, diabetic gastroparesis or postoperative ileus. Other disease wherein the therapeutic system of the present invention is used for treatment are sepsis and organ transplantation, kidney transplantation, sickle cell anemia, hypertension, idiopathic pulmonary fibrosis, COPD or respiratory distress syndrome.

In one embodiment, the therapeutic system comprises a compound C which enhances therapeutic gas release from A when the therapeutic system is administered to a patient. Compound C is different from compound B and can be a carbonyl substituting ligand, a sulfur-compound, an acid or base, an enzyme, a redox reagent, or a light-emitting compound.

Preferably compound C is an acid or base, such a citric acid. If gas release from A is e.g. pH dependent, an acid or base can be comprised in the therapeutic system which creates a certain pH in the local environment of the gas releasing molecule upon application of the therapeutic system to the patient. Thereby, gas release is triggered from compound A when the therapeutic system is administered to the patient. Gas release is thus not dependent on the pH of a certain tissue in the body, but the gas releasing trigger is delivered with the therapeutic system so that the gas release becomes independent of environmental pH.

The present invention also relates to a coated tablet for oral administration comprising compounds A, B and preferably also a compound C, wherein A is a CO releasing molecule, B is a sulfur compound and C, if present, is an acid, wherein B is comprised in the tablet as coated particles.

The therapeutic system releases a therapeutic gas in a therapeutically effective amount when administered to a patient. In one embodiment, the therapeutic system of the present invention releases between 0.1 and 100 μmol CO when administered to a patient, preferably between 1 and 50 μmol. Preferably, the therapeutic gas release from the therapeutic system of the present invention is controlled in that therapeutic gas is released from the system over a prolonged time period after administration of the system to the patient. In one embodiment, therapeutic gas is released from the therapeutic system after administration to the patient for at least two, three or four hours.

In one embodiment, particularly the metal of the metal carbonyl complex is retained in the therapeutic system. It is thus entrapped in the therapeutic system so that it is not released in the patient's body upon administration of the therapeutic system to the patient. The metal is e.g. excreted after oral administration of the therapeutic system in form of a tablet with the essentially intact tablet from which the therapeutic gas was, however, released in the gastrointestinal tract upon administration to the patient.

The present invention also relates to the use of a sulfur compound in a tablet comprising compound A wherein A is a CO releasing molecule for enhancing gas release from A when the tablet is administered to a patient.

The examples hereafter are intended to illustrate the invention without however limiting it.

### Example

An easy to use tablet referred to as oral carbon monoxide release system (OCORS) providing precise, controlled, tunable and targeted CO delivery for the treatment of sequelae of gastrointestinal diseases was prepared. OCORS is an oral tablet based on sulfite induced CO release from the CO releasing molecule 2 (CORM-2, Tricarbonyldichlororuthenium(II) dimer; [Ru(CO)₃Cl₂]). OCORS performance was detailed as a function of the presence of buffer within the tablet core and the composition of a semipermeable cellulose acetate coating, shielding the tablet core. OCORS delivered CO for up to 10 hours with zero order release within approximately 30 to 240 minutes. This controlled release system delivered CO independent of environmental pH, such that the therapeutic gas can be reliably generated at gastric, intestinal or colonic sites.

### Amperiometric Detection System

An Erlenmeyer flask purchased from Gebr. Rettberg (Gottingen, Germany) was used as reaction space. For insertion of the CO sensor and to seal the reaction space, a conically tapered joint from Schott Medica (Wertheim, Germany) was equipped with a glass guide tube for an electric wire. As a prerequisite for cross referencing with calibration gas, a single-bore stopcock was connected to the Erlenmeyer flask as well as to the conically tapered joint. The integrated CO sensor of the “Ei207D” CO detector (Ei Electronics, Shannon, Ireland) was removed and externally connected using a “Wire Wrap” 0.404 mm² wire (Kabeltronik, Denkendorf, Germany) linked with a 1.3 mm accessory shoe (Vogt AG, Lostorf, Switzerland). The wire was glued into the guide tube with “UHU plus endfest” epoxide resin (UHU, Bũhl, Germany) to seal the system. On-line videos were collected monitoring the detector with USB webcams with the Eyeline video surveillance software (NCH Software, Canberra, Australia).

### Measurements of CO Release

### CORM-2 Suspension

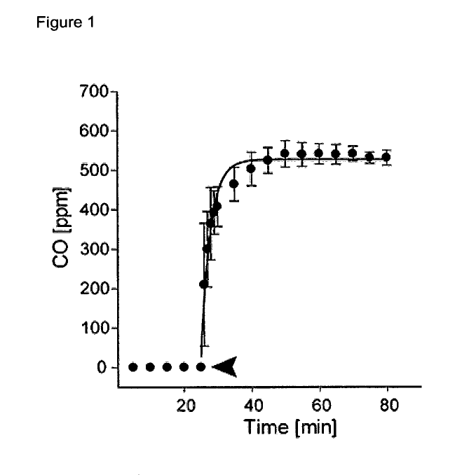

The CO release from CORM-2 was triggered by Na₂SO₃ and amperiometrically detected. For that, 4.3 mg CORM-2 were placed in the Erlenmeyer flask filled with 15 ml Millipore water and stirred at 130 rpm (Variomag Telesystem, Thermo Scientific, MA). After 25 minutes, 10 mg Na₂SO₃ were added to the reaction space. CO release in ppm as read from the detector of each system was calibrated with 100 ppm CO calibration gas (diluted in air; from Real Gas (Martinsried, Germany) and normalized, accordingly (see FIG. 1 for results).

Calibration of CO Release from CORM-2

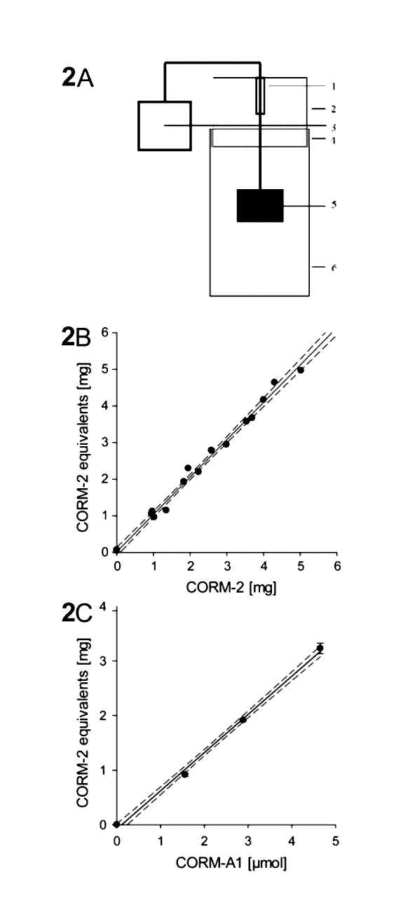

A concentration series of CO was generated by transferring different amounts of CORM-2 into 25 mL of a stirred (130 rpm) 333 mg/L aqueous Na₂SO₃ solution. CO release [ppm] after 60-80 minutes was plotted against the amount of CORM-2 [mg] placed into the system. Following this procedure, CO release [ppm] is expressed as amount of CORM-2 [mg], with one CORM-2 equivalent being defined as the amount of CO released per milligram CORM-2 after 60-80 minutes and when exposed to the experimental conditions described in this section (see FIG. 2B for results). CO release from CORM-2 expressed in CORM-2 equivalents [mg] followed a linear pattern (y=0.0192+1.0199x; r²>0.99; n=1 for 14 independent measurements).

Calibration of CO Release from CORM-A1

The calibration of CO release using CORM-A1 (sodium boranocarbonate; Na2[H₃BCO₂]) was modified from previous reports [23]. In brief, a concentration series of CO was generated by diluting different amounts of CORM-A1 in ice water and transferring this solution to 25 mL of a stirred (130 rpm) citric acid buffered solution (pH 5.5) at room temperature. Release of CO expressed as CORM-2 equivalents [mg] (vide supra) was recorded as a function of CO release from different amounts of CORM-A1 [μmol]. Thereby, CO release from CORM-A1 was linked to CO release from CORM-2 and, consequently, CO release from CORM-A1 could now be expressed in CORM-2 equivalents [mg] by means of linear regression (y=−0.0776+0.6996x; r²>0.99; (n=3).

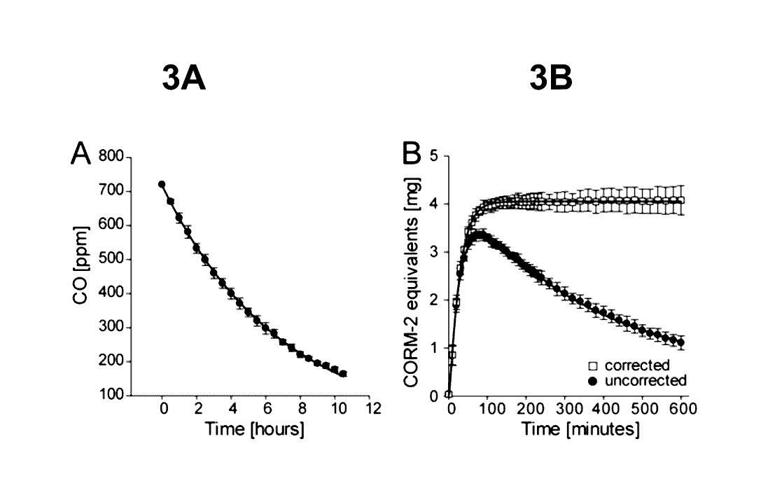

CO data reported in the example is calculated from the corresponding release from CORM-2 equivalents and if reported for CORM-A1, calculated using linear regression. The sensor is destructive to CO (oxidizes CO to CO₂) and hence consumes the analyte. Therefore, CO loss by and from the system was quantified and basis for data correction. For the correction, the loss by and from the system was recorded and added to the data as read from the detector (vide infra). For assessment of CO loss and data correction, CO was generated from CORM-2 in presence of Na₂SO₃ with stirring at 130 rpm, until reaching a value of approximately 800 ppm. At this point the stirring was stopped. CO loss [ppm] was recorded when a value of 720 ppm was reached and followed an exponential decline (y=717.5*e−0.145x; r²>0.99; n=5; see FIGS. 3A and 3B for results).

Na₂SO₃ Crystal Collection and Crystal Coating

Na₂SO₃ crystals of appropriate size were collected using an AS 200 Retsch analytical sieve tower (Haan, Germany) and the 250-500 μm fraction was collected. These crystals were coated using solutions consisting of 8.6 g Eudragit E PO (from Evonik Industries, Essen, Germany), 0.9 g sodium dodecyl sulfate, 1.3 g stearic acid, 4.3 g talcum, 50 mL of distilled water and 50 mL of absolute ethanol. The dye Sam specracol erythrosine Ik was added in minute amounts to visualize the coating with the overall recipe following the manufacturer's instructions [24]. The preparation was homogenized for 20 min at 13,000 rpm using a Silent Crusher M (Heidolph, Schwabach, Germany) and sieved through a mesh with 375 μm aperture size for removal of disruptive agglomerates. 60 g Na₂SO₃ crystals were coated with a Mini-Coater (Glatt, Binzen, Germany) used in top-spray configuration at a temperature of 45° C., an atomizing pressure of 0.86 bar. The coating solution was pumped into the coater by a Flocon 1003 flexible tube pump (Roto-Consulta, Lucerne, Switzerland) at 0.7 mL/min. Coating lasted for about 2 hours and the fluidized bed was maintained for another 10 minutes, thereafter. Na₂SO₃ content of the coated crystals was 86% as quantified by the compendial method for iodometric determination [25]. Scanning electron microscopy (SEM) was used to assess the homogeneity of the coating of Na₂SO₃ crystals and the distribution of Na₂SO₃ crystals within the tablet. Samples were sputter coated with palladium/gold prior for evaluation on a JEOL JSM 7500F scanning electron microscope (Tokyo, Japan) at an accelerating voltage of 5 kV using lower secondary electron signals.

### Development of the Oral Carbon Monoxide Release System (OCORS)

### Preparation of the Tablets

Tablets were prepared from a blend of 72 mg pulverized citric acid*H₂O, 128 mg pulverized trisodium citrate*2 H₂O, 200 mg coated Na₂SO₃ (vide supra), 60 mg CORM-2 and 1.54 g tableting mixture (consisting of lactose, cellulose, aluminium oxide and magnesium stearate, purchased from Meggle, Wasserburg am Inn, Germany) prepared for 30 minutes in a Turbula T2F mixer (WAB AG, Muttenz, Switzerland). The resulting blend was transferred into an eccentric press tableting machine model FE136SRC from Korsch (Berlin, Germany) using a 7 mm tablet punch from Korsch (Berlin, Germany) resulting in average tablets weights of 120 mg. Non-buffered tablets were prepared by replacing the amount of citric acid buffer mentioned above by the tableting mixture. A tablet coating solution was prepared from 0.9 g PEG 400 as pore former in 100 mL acetone (1×pore former PEG 400; vide infra) into which 5.8 g cellulose acetate (Eastman cellulose acetate CA398-10NF/EP was from Gustav Parmentier GmbH (Frankfurt am Main, Germany) was slowly added under stirring at 130 rpmln another set of experiments, 9 g of PEG 400 was introduced (10×pore former PEG 400; vide infra). The tablet cores were dip coated in the cellulose acetate coating. For that, the cores were completely immersed into the coating solution and subsequently air dried using an air gun at about 60° C. for 1 min. Thereafter, the pre-dried sample was transferred into a desiccation and left in an ED 53 drying chamber (Binder, Tuttlingen, Germany) at 50° C. for 30 min. Tablet cores were coated once (10×pore former PEG 400 used), four or eight times (1×pore former PEG 400 used). The structure of the tablet core and the coating were assessed following palladium/gold sputter coating and by a JEOL JSM 7500F scanning electron microscope (Tokyo, Japan) at an accelerating voltage of 5 kV using lower secondary electron signals.

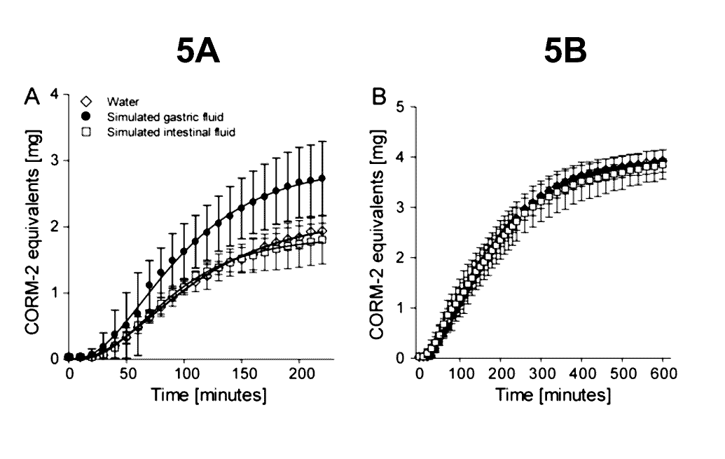

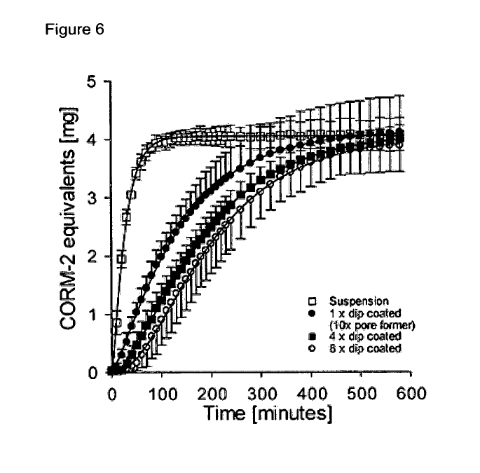

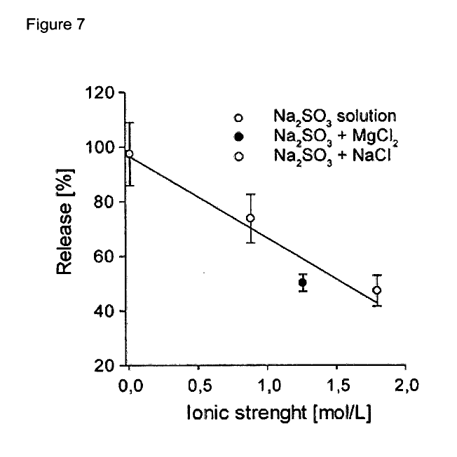

CO release from the oral carbon monoxide release system (OCORS) Coated tablets were transferred into 25 mL of distilled water, compendial simulated gastric fluid without enzymes (prepared as described in USP 37 [26]) or simulated intestinal fluid without enzymes (prepared as described in USP 37 [26]). Experiments assessing the impact of the release medium on CO release (see FIG. 5A, 5B) were done in comparison to control experiments, within which 12 mg coated Na₂SO₃ crystals were measured in simulated gastric fluid without enzymes to control for possible detector interference with sulfites (interference was not observed with these experimental conditions; data not shown). Experiments assessing the impact of the coating procedure on CO release were done in comparison to control experiments, within which 3 mg CORM-2 was suspended in 25 mL of 0.333 g/L Na₂SO₃ solution (see FIG. 6 for results). In another set of control experiments, the impact of the ionic strength of the release medium and the impact of the salt with which the ionic strength was set (using sodium chloride solutions to set ionic strengths of 0.88 mol/L and 1.8 mol/L, respectively, and magnesium chloride to set an ionic strengths of 1.26 mol/L) on CO release was studied using CORM-2 suspensions. For that, CORM-2 was exposed to 25 mL of 0.333 g/L Na₂SO₃ in the appropriate release medium (for results, see FIG. 7).

### Statistics

All data were reported as mean±standard deviation unless specified otherwise. Statistical significance for ruthenium analytics was calculated by Student's t-test for pairwise comparison and linear regression was made using conventional software packages (Sigma Plot, Systat Software Inc., CA). One-Way ANOVA was used for determination of release pattern and Tukey test for post-hoc comparison (Minitab-statistics, Coventry, UK). p<0.05 was considered statistically significant.

### Results

Sulfite Triggered Release from CORM-2

CO release of CORM-2 in the presence of water was studied. CORM-2 suspended in water demonstrated no detectable CO release (FIG. 1). After 25 min, Na₂SO₃ was added triggering instantaneous CO release, which was completed within 25 minutes, and plateauing at 550 ppm after 40 minutes (FIG. 1).

### Calibration Procedure

The amperiometric sensor was connected to the CO detector and continuously monitored the headspace within a sealed Erlenmeyer flask, within which the CO release experiments were conducted (FIG. 2A). CO release from CORMs was expressed in CORM-2 equivalents, with one CORM-2 equivalent being defined as the amount of CO released per milligram CORM-2 and when exposed to 25 mL of a stirred (130 rpm), 333 mg/L aqueous Na₂SO₃ solution for 60-80 minutes. The time frame of 60-80 minutes was sufficient to ensure >90% release (FIGS. 1, 3A, 3B). CO release from different amounts of CORM-2 directly correlated to the CORM-2 equivalents, indicating the suitability of the amperiometric platform and the normalization method deployed here within for the assessment of CO release profiles (FIG. 2B). This calibration procedure was instrumental for normalizing the three amperiometric systems used in parallel for the experiments. The regression of the CO released from different amounts of CORM-A1 against CORM-2 equivalents indicated a linear relationship with a slope of approximately 0.7 (FIG. 2C). Data correction was necessary to further refine the data and reflecting the destruction of CO by the sensor (oxidizes CO to CO₂), and hence consumption of the analyte or possible escape from the system. For that, the actual released amount was calculated by correcting the data as read from the display by the CO loss curve (FIG. 3A). CO consumption over time started at 720 ppm and declined to 150 ppm within 10.5 hours, following an exponential decline (FIG. 3A). The impact of data correction was outlined by control experiments using sulfite triggered CO release from CORM-2 (FIG. 3B). Without correction, CO release peaked at approximately 60-80 min followed by a subsequent decline reflecting CO consumption by the sensor or loss of CO from system, ultimately declining to a third of its peak value after 8.5 hours. Applying the correction function (FIG. 3A) plateauing at approximately 140 minutes was observed and no decline, thereafter (FIG. 3B).

### Development of the Oral Carbon Monoxide Release System (OCORS)

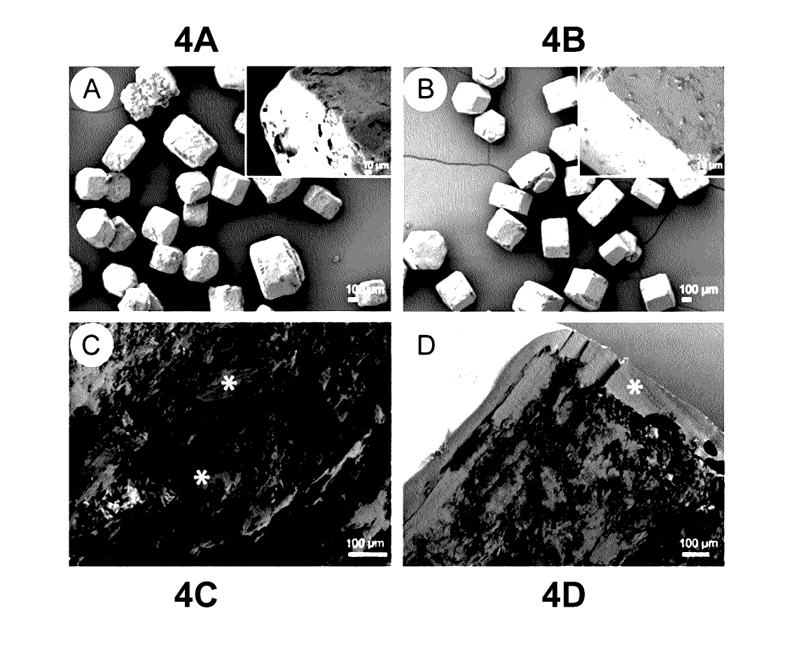

OCORS is a CO release system, controlled by water permeation through the semipermeable cellulose acetate shell. The permeating water causes swelling of the coating around the coated sodium sulfite crystals within the tablet core and rapid dissolution, thereof. Thereby, dissolved sodium sulfite gets access to the CORM-2, sparking CO release. Cellulose acetate coating was used to control the flux of water into the system. The coating around the sodium sulfite crystals was instrumental in separating it from CORM-2 in the solid state and as a prerequisite to allow sustained storage of OCORS without CO release. The coating of the sodium sulfite crystals resulted in smooth films covering the entire crystal surface (FIG. 4A, 4B) and coated crystals were homogenously distributed throughout OCORS (FIG. 4C). OCORS's shell consisted of a smooth cellulose acetate coating (FIG. 4D). When OCORS was prepared from powder blends with no citric acid buffer, CO release was affected by the release media, with exposure to simulated gastric fluids (pH 1.2) resulting in a trend to higher CO release as compared to water or simulated intestinal fluid (pH 6.8; FIG. 5A). The sensitivity to environmental parameters was addressed by blending citric acid buffer into the powder blend before tableting, yielding an overall higher CO release as compared to tablets prepared without buffer and indistinguishable release profiles in all three selected release media (FIG. 5B). The OCORS release pattern was in three distinct consecutive phases, with phase (i) up to 30, phase (ii) 30 to 240, and phase (iii) exceeding 240 minutes, respectively. The second phase was characterized by a linear relationship of CO release and time (y=0.017*t0.98; r²=0.9), whereas the third phase deviated from the linear relationship and asymptotically plateaued at about a total CO release of 4 CORM-2 equivalents.

CO release from CORM-2 suspensions was impacted by ionic strength but not by the salt with which the ionic strength was set (FIG. 7). Increasing the ionic strength from 0.008 mol/L in Na₂SO₄ solution to 0.88 mol/L using NaCl reduced CO release by 26±9% (n=3). An ionic strength of 1.26 mol/L set with MgCl₂ significantly reduced the ionic strength by 50±3% (n=3). Likewise an ionic strength of 1.8 mol/L set with NaCl significantly reduced the CO release by 53±6% (n=3); (FIG. 7). In order to further control the CO release rate, the permeability through OCOR's shell was modified by (i) varying the concentration of pore former and (ii) the coating thickness (FIG. 6). CORM-2 suspension used for control demonstrated half maximal release after 20 minutes. Likewise half maximal release was reached within 175±33 minutes when 8 times dip coated, 157±16 minutes when 4 times dip coated and 103±15 minutes when 1 time dip coated (the solution used for 1 time dip had tenfold concentrations of the pore former PEG 400in the coating solution as compared to the solutions used for the 4 times and 8 times dip coating, respectively). Results were statistically significant for the difference between 8 and 1 time coating and between the 4 and 1 time coating, respectively (p<0.001; n=6). CO release was completed from all coated systems within approximately 10 hours (FIG. 6). The dimensions and appearance of OCORS remained unchanged throughout the study.

1. A therapeutic system comprising compounds A and B, wherein:
A is a therapeutic gas releasing compound, A and B are not in contact with each other in the therapeutic system during storage, and B is a compound that enhances therapeutic gas release from A when the therapeutic system is administered to a patient,
further wherein:
A is a carbon monoxide releasing molecule and B is a sulfur compound,
A is selected from the group consisting of CORM-2, CORM-3, and CORM-401, and
the therapeutic system is a tablet of capsule.

2. (canceled)

3. The therapeutic system according to claim 1, wherein A in B come in contact with each other when the therapeutic system is administered to a patient.

4. The therapeutic system according to claim 1, wherein A and B are comprised in the therapeutic system as A particles and B particles and contact of A with B is prevented in the therapeutic system during storage by a coating around the A and/or B particles.

5. The therapeutic system according to claim 1, wherein the therapeutic system is formulated for oral administration.

6. (canceled)

7. The therapeutic system according to claim 1, wherein the therapeutic system is coated.

8. (canceled)

9. The therapeutic system according to claim 1, comprising compound C which enhances therapeutic gas release from A when the therapeutic system is administered to a patient.

10. (canceled)

11. (canceled)

12. Use of a sulfur compound in a tablet comprising compound A for enhancing gas release from A when the tablet is administered to a patient, wherein A is a carbon monoxide releasing molecule and wherein the sulfur compound and the carbon monoxide releasing compound are not in contact with each other in the therapeutic system during storage, and wherein CORM-2, CORM-3, or CORM-401 is used as compound A.
